(19) United States
(12) Patent Application Publication
(10) Pub. No.: US 2016/0287107 A1
(43) Pub. Date: Oct. 6, 2016

(54) INTELLIGENT PHOTOPLETHYSMOGRAPH SIGNAL-TO-NOISE RATIO CONTROL FOR RECOVERY OF BIOSIGNALS DURING TIMES OF MOTION

(71) Applicant: INTEL CORPORATION, Santa Clara, CA (US)

(72) Inventors: Steven P. Szabados, San Francisco, CA (US); Andrew A. Stirn, San Francisco, CA (US)

(21) Appl. No.: 15/038,165

(22) PCT Filed: Jan. 30, 2015
(86) PCT No.: PCT/US2015/013664

§ 371 Date: May 20, 2016

Related U.S. Application Data
(60) Provisional application No. 61/933,745, filed on Jan. 30, 2014.

(51) Int. Cl. A61B 5/024; A61B 5/08; A61B 5/00

(52) U.S. Cl. CPC A61B 5/02416; A61B 5/681; A61B 5/721; A61B 5/0816; A61B 5/7278; A61B 5/02438

(57) ABSTRACT

An optical pulse rate detector includes at least one emitter, a photodetector, and an adaptive filter. The emitter transmits light at a first light output level and a second light output level into body tissue of a user. The photodetector receives reflected light from the body tissue and produces a voltage indicative of an amount of the reflected light. The adaptive filter receives a first output signal from the photodetector indicative of an amount of the light at the first light output level reflected from the body tissue and a second output signal indicative of an amount of the light at the second output level reflected from the body tissue. The adaptive filter removes the second output signal from the first output signal to generate a pulse rate signal indicative of a pulse rate of the user.

### CROSS-REFERENCE TO RELATED APPLICATIONS

This application claims the benefit of U.S. Provisional Patent Application No. 61/933,745, filed Jan. 30, 2014, which is incorporated herein by reference in its entirety.

### BACKGROUND

Optical pulse rate detectors measure pulse rate of users by detecting light reflected from a user's skin. Optical pulse rate detectors are often incorporated into wearable devices for continuously monitoring the user. However, if a user is moving while wearing the wearable device, the amount of light reflected to the optical detector may vary from sample to sample. Thus, during periods of motion, the optical pulse rate detector detects motion artifacts that contaminate the pulse rate signal.

Some wearable devices use a second sensor, such as an accelerometer, to measure the motion of a wearer of the device. However, as the orientation of the wearable device (and thus the orientation of the accelerometer), stride patterns, and so forth vary from person to person, the signal generated by an accelerometer is not directly correlated to the motion artifacts measured by an optical pulse rate detector. Thus, it is difficult to remove the motion artifacts using a second sensor.

### BRIEF DESCRIPTION OF THE DRAWINGS

The disclosed embodiments have other advantages and features which will be more readily apparent from the detailed description, the appended claims, and the accompanying figures (or drawings). A brief introduction of the figures is below.

FIG. 1 illustrates a wearable device, according to one embodiment.

FIG. 2 illustrates an alternative view of a wearable device, according to one embodiment.

FIG. 3 illustrates another view of a wearable device, according to one embodiment.

FIG. 4 is a schematic illustrating components of an optical pulse rate detector, according to one embodiment.

FIG. 5 illustrates operational waveforms of the optical pulse rate detector, according to one embodiment.

### DETAILED DESCRIPTION

The Figures (FIGS.) and the following description relate to preferred embodiments by way of illustration only. It should be noted that from the following discussion, alternative embodiments of the structures and methods disclosed herein will be readily recognized as viable alternatives that may be employed without departing from the principles of what is claimed.

Reference will now be made in detail to several embodiments, examples of which are illustrated in the accompanying figures. It is noted that wherever practicable similar or like reference numbers may be used in the figures and may indicate similar or like functionality. The figures depict embodiments of the disclosed system (or method) for purposes of illustration only. One skilled in the art will readily recognize from the following description that alternative embodiments of the structures and methods illustrated herein may be employed without departing from the principles described herein.

### Configuration Overview

An optical pulse rate detector is configured to measure pulse rate of a user while the pulse rate detector is moving. The optical pulse rate detector takes two successive samples per sampling period: one to sample the pulse of the user, and one to sample motion of the user. Using an adaptive filter, the optical pulse rate sensor removes motion artifacts from the measured pulse rate to increase the signal-to-noise ratio of the pulse rate signal. Because the optical pulse rate detector measures both the user's pulse rate and the user's motion, the measured motion more closely approximates the motion artifacts in the detected pulse rate signal than motion measured by an external sensor. In one embodiment, the pulse rate detector is a component of a wearable device monitoring physiological and kinematic parameters of a wearer of the device.

### Example Wearable Device

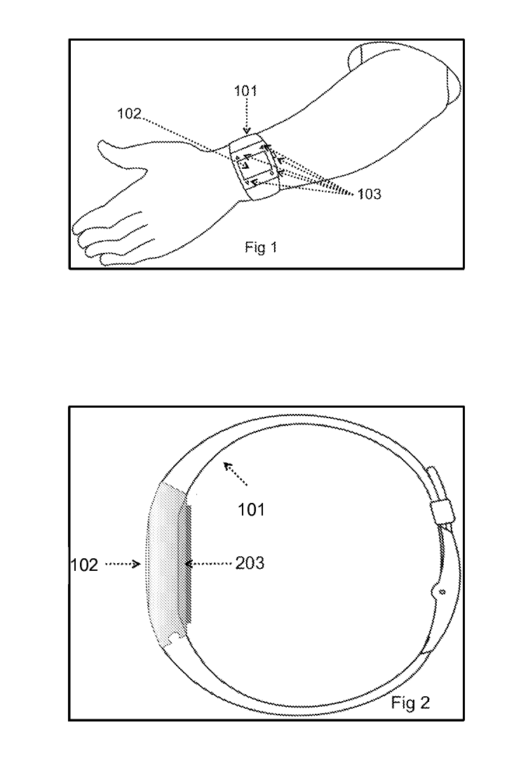

FIG. (FIG. 1 illustrates an example of a wearable device 100 configured to be in close proximity to or in contact with a user. For example, the device 100 may be worn on a user's appendage or portion thereof, such as an arm or a wrist. A fastening system 101 fastens the device 100 to a user's appendage. The fastening elements 101 may be removable, exchangeable, or customizable. Furthermore, although embodiments are described herein with respect to a wrist-worn device, other form factors or designed wear locations of the wearable device 100 may alternatively be used. For example, embodiments of the method described herein may be implemented in arm-worn devices, head-worn devices, clip-on devices, and so forth. Furthermore, the various components of the device 100 described herein may alternatively be components of two or more devices communicatively coupled by wired or wireless communication, rather than enclosed within a single device. In one embodiment, the wearable device 100 is a physiological monitoring device for monitoring activities of its wearer and calculating various physiological and kinematic parameters, such as activity levels, caloric expenditure, step counts, heart-rate, and sleep patterns.

The wearable device 100 includes a display (or screen) 102 and several user interaction points 103. The display 102 and user interaction points 103 may be separate components of the device 100, or may be a single component. For example, the display 102 may be a touch-sensitive display configured to receive user touch inputs and display information to the user. The wearable device may also have a display element such as 102 without interaction points, or interaction points 103 without a display element such as 102. The interaction points 103 used by the user to interface with the device and may be, for example, physical buttons, solid state touch sensitive sensors, a separate touch sensitive display or dedicated regions of the display 102.

It should be noted that the device 100 may include additional components not shown in FIG. 1. In particular, the device 100 includes one or more sensors for monitoring various physiological or kinematic parameters of the wearer of the device 100, for example, pulse rate, blood flow, body temperature, and motion.

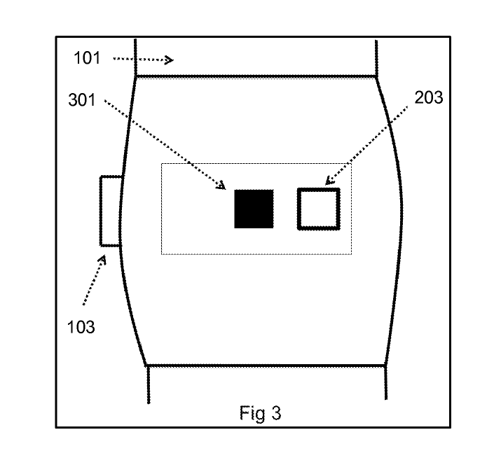

FIG. 2 is a side view of an embodiment of the device 100, showing a fastening system 101, a display (or screen) 102, and one or more processors (generally, processor 203). Another view of an embodiment of the wearable device 100 is shown in FIG. 3. FIG. 3 shows a view from beneath the device 100, illustrating the fastening mechanism 101, the processor 203, an optical pulse rate detector 301, and one or more user interaction points 103 visible from beneath.

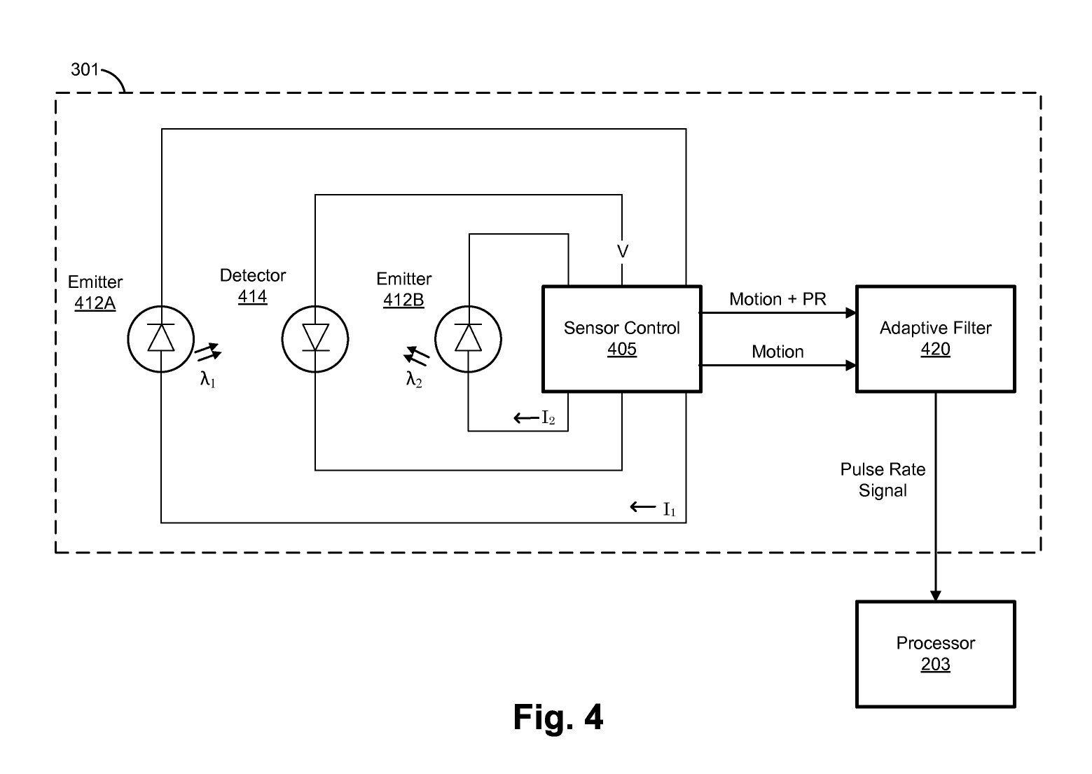

The amount of light reflected to the optical pulse rate detector 301 depends in part on the orientation of the device 100. Thus, as a wearer of the device 100 is moving, the amount of light reflected to the optical pulse rate detector 301 is a function of not only the volume of blood beneath the wearer's skin, but also the wearer's movements. The optical pulse rate detector 301 is configured to measure a baseline reflectance of the wearer's skin in addition to measuring the blood volume. The baseline reflectance represents an amount of light reflected by the skin independent of the volume of blood beneath the skin. Using the baseline reflectance, the optical sensor 301 removes motion artifacts from the detected blood volume to generate data indicative of the pulse rate of the wearer. The optical sensor 301 is described further with respect to FIG. 4.

The processor 203 is communicatively coupled (e.g., via a data bus) to the optical pulse rate detector 301 for processing the pulse rate data captured by the optical pulse rate detector 301. Using the pulse rate data received from the optical pulse rate detector 301, the processor 203 generates biometric data about the wearer of the device 100, such as pulse rate, beat-to-beat variance, respiration, beat-to-beat magnitude, and beat-to-beat coherence. The processor 203 is also communicatively coupled to the display 102 for controlling the display 102. Under the control of the processor 203, the display 102 displays various pieces of information to a user, such as the biometric data generated by the processor 203. Although the processor 203 is shown in FIG. 3 as being integrated into the device 100, in other embodiments the processor 203 is external to the device 100.

### Example Optical Pulse Rate Detector

Referring now to FIG. 4, illustrated is a schematic of the optical pulse rate detector 301 configured to measure a pulse rate signal in the presence of body motion. In one embodiment, the optical pulse rate detector 301 includes a sensor control 405, one or more emitters (generally, 412), a photodetector 414, and an adaptive filter 420.

The emitters 412A, B and photodetector 414 are configured to be placed in proximity to the skin of a wearer (or user) of the device 100, such that the emitters 412 emit light onto the skin of the wearer and the photodetector 414 measures light reflected from the skin of the wearer. In the embodiment illustrated in FIG. 4, the optical pulse rate detector 301 includes two emitters 412A, 412B. Each emitter 412A, 412B may be configured to emit monochromatic light, or may be configured to emit light of more than one wavelength. For example, the emitters 412A, 412B may be light emitting diodes (LEDs). In one embodiment, the emitter 412A is configured to emit light of a wavelength λ₁ that is responsive to blood flow (e.g., green light), while the emitter 412B is configured to emit light of a wavelength λ₂ that is responsive to motion (e.g., amber light). In another embodiment, the optical pulse rate detector 301 has a single emitter 412. The photodetector 414 measures intensity of the light reflected from the skin of the wearer and converts the measured intensity into a voltage, V, which is input to the sensor control 405.

The sensor control 405 is a controller configured to receive, process, and transmit signals. For example, the sensor control 405 is configured to send a periodic current signal to one or both emitters 412 of the optical pulse rate detector 301 and receive a voltage signal from the photodetector 414 indicating an amount of light reflected from the skin of the wearer. When an emitter 412 emits light of a wavelength that is responsive to blood flow, the voltage signal received from the photodetector 414 is indicative of a volume of blood beneath the skin of the wearer. Accordingly, the sensor control 405 samples the pulse rate of the wearer by sending a current pulse to an emitter 412. If light from an emitter 412 is not as readily absorbed by blood, the amount of light detected by the photodetector 414 (and the voltage signal generated thereby) is a baseline reflectance. The change in the baseline reflectance from one sample to the next is indicative of an amount of motion of the optical pulse rate detector 301.

The sensor control 405 drives the optical pulse rate detector 301 to produce two samples at different light outputs. A first light output from the optical pulse rate detector 301 is responsive to blood flow, while a second light output is responsive to motion of the user of the device 100. For example, the first light output is light having a wavelength readily absorbed by blood and/or light having a higher intensity than the second light output. The second light output is, for example, light having a wavelength that is not as readily absorbed by blood as the first light output. The sensor control 405 samples the volume of blood beneath the wearer's skin using the first light output and samples the baseline reflectance of the skin of the wearer using the second light output.

In embodiments of the device 100 including two emitters 412A, 412B, the sensor control 405 generates a current signal I₁ to drive emitter 412A to sample the blood volume. The current signal I₁ includes a series of pulses that cause the emitter 412A to emit a first light signal at wavelength λ₁ and an intensity proportional to the magnitude of the pulses. At each pulse, the photodetector 414 detects an amount of the first light signal reflected by the wearer's skin and generates a voltage V, which is received by the sensor control 405.

Similarly, to sample the baseline reflectance, the sensor control 405 generates a current signal I₂ to drive emitter 412B. The current signal I₂ includes a series of pulses that cause the emitter 412B to emit a second light signal at wavelength λ₂, where the intensity of the second light signal is also proportional to the magnitude of the pulses. At each pulse of the current signal I₂, the photodetector 414 detects an amount of the second light signal reflected by the wearer's skin and generates a voltage V input to the sensor control 405.

In some cases, the magnitudes of the pulses of the current signals I₁ and I₂ may be the same, or the wavelengths λ₁ and λ₂ may be the same. For example, the sensor control 405 may drive both emitters with current signals having pulses with equal magnitude while sampling the blood volume using light at wavelength λ₁ and the baseline reflectance using light at wavelength λ₂ (in which λ₁≠λ₂). As another example, both emitters may emit light at wavelength λ₁ and the sensor control 405 drives the emitters with current signals I₁ and I₂ to sample the blood volume and baseline reflectance, respectively, in which the magnitude of the pulses of I₁ is greater than the pulse magnitude of I₂. In other cases, the blood volume and baseline reflectance are sampled using light having both different intensities and different wavelengths λ₁ and λ₂. In embodiments of the device 100 having a single emitter 412, the sensor control 405 drives emitter 412 to produce a first light output using a current signal I₁ having a first magnitude, and drives emitter 412 to produce a second light output using a current signal I₂ having a second magnitude that is lower than the first magnitude.

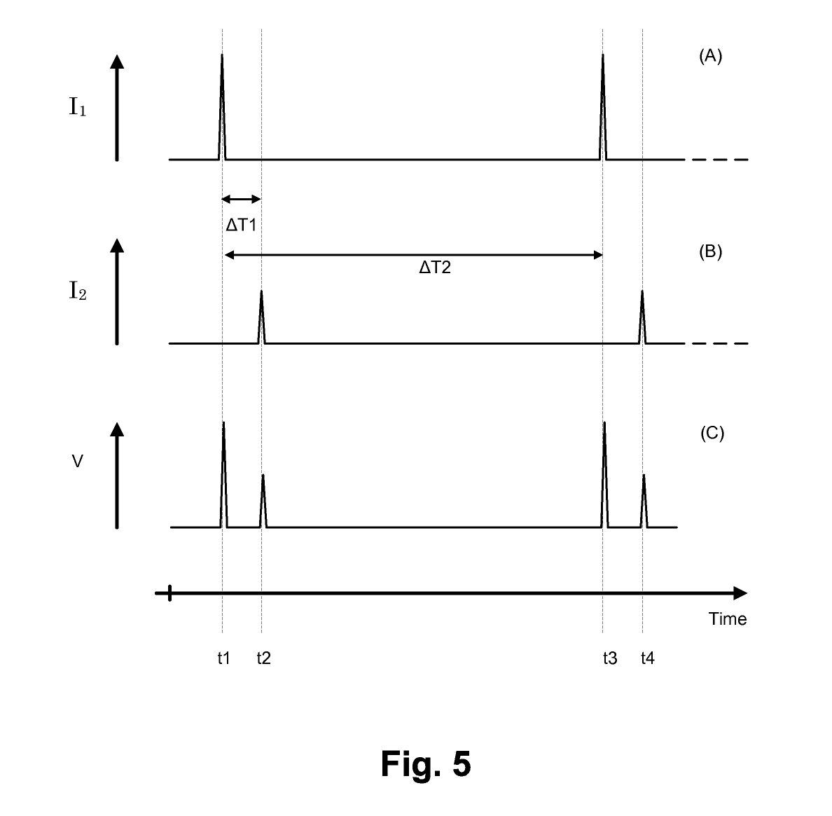

Referring to FIG. 5, it illustrates graphically example drive signals I₁ and I₂ generated by the sensor control 405 to drive the emitters 412, as well as the voltage output by the photodetector 414. In the three graphs shown, (A) and (B) show current in along the y-axis and time along the x-axis. The graph at (C) shows voltage along the y-axis and time along the x-axis.

As shown in FIG. 5, the sensor control samples the blood volume at time t1, t3, etc., and samples the baseline reflectance at time t2, t4, etc. The interval between a sample of the blood volume and a sample of the baseline reflectance is given by ΔT1=t2−t1, and the interval between two successive samples of the blood volume is given by ΔT2=t3−t1. The interval ΔT2 defines the sampling period of the wearer's pulse rate, and can be adjusted to achieve a desired sampling frequency.

In one embodiment, the sensor control 405 samples the baseline reflectance shortly after sampling the blood volume, such that ΔT1<ΔT2. When ΔT1 is small, the baseline reflectance closely approximates the actual baseline reflectance at the time the pulse rate was sampled. The interval ΔT1 may be selected based on properties of the emitters 412, the photodetector 414, or other hardware of the optical sensor 301. For example, ΔT1 is selected based on the response time of the photodetector 414 to ensure the response of the photodetector 414 to the baseline reflectance sample is distinct from the response to the blood volume sample. It is noted that rather than sampling the baseline reflectance after sampling the blood volume, the sensor control 405 may alternatively sample the baseline reflectance first. As also shown in FIG. 5, V (graph (C)) represents the voltage output by the photodetector 414 in response to both the first light output sample and the second light output sample.

The optical pulse rate detector 301 measures pulse rate of a user by measuring volume of blood in a given area over time. An emitter of the optical pulse rate detector 301 sends a light signal to skin and tissue of the wearer of the device 100 and measures the amount of light reflected to a photodetector. A portion of the light signal emitted by the emitter is absorbed by the wearer's tissue and a portion is reflected to the photodetector. In particular, if the light is of a wavelength absorbed by blood, a portion of the light is absorbed by the blood of the wearer of the device. Thus, the amount of light reflected to the photodetector depends in part on the volume of blood under the skin. As blood volume in the measured area cyclically changes due to cardiac cycles, the amount of light detected by the photodetector cyclically varies. The photodetector converts the measured light intensity into a voltage, which is analyzed for regular variations that indicate the heart's pulsation of blood throughout the body of the wearer.

Returning to FIG. 4, the sensor control 405 converts the voltage V received from the photodetector 414 to digital samples of the baseline reflectance and blood volume, and generates two output signals: Motion and Motion +Pulse Rate (PR). The Motion +PR signal is a function of pulse and a function of motion, comprising the digital samples of the voltage V generated by the photodetector 414 in response to the first output light signal. Thus, the Motion +PR signal is a sequence of samples of the blood volume beneath the wearer's skin, but includes motion artifacts. The Motion signal is a function of the wearer's motion and comprises a sequence of samples of the voltage, V, generated by the photodetector 414 in response to the second light output signal, corresponding to samples of the baseline reflectance of the skin of the wearer.

In one embodiment, the sensor control 405 is also configured to determine the second light output based on the voltage received from the photodetector 414. The sensor control 405 compares the Motion +PR signal to the Motion signal, and adjusts the magnitude of the pulses of the current signal I₂ driving the emitter 412B based on the comparison. For example, if a correlation between the Motion +PR signal and the Motion signal is greater than a threshold, the sensor controller 405 decreases the pulse magnitude of the drive signal I₂. Accordingly, the sensor control 405 adjusts the magnitude of the pulses of I₂ to reduce the dependency of the Motion signal on the volume of blood beneath the skin of the wearer.

The adaptive filter 420 receives the signals Motion and Motion+PR from the sensor control 405. Using the Motion signal as an indicator of the wearer's motion, the adaptive filter 420 removes the motion artifacts from the Motion+PR signal to generate a pulse rate signal. In various embodiments, the adaptive filter 420 may implement a least mean squares algorithm, a recursive least squares algorithm, or another type of adaptive filter algorithm. The adaptive filter 420 sends the derived pulse rate signal to the processor 203, which analyzes the signal to determine pulse rate of the wearer of the device 100.

By removing the motion of the wearer from the detected pulse rate signal, the adaptive filter 420 increases the signal to noise ratio of the pulse rate signal. In addition, motion measured by the same sensor as used to measure pulse rate more closely approximates the motion artifacts in the detected pulse rate signal than motion measured by an external sensor. As a result, the adaptive filter 420 improves the accuracy of the pulse rate determined by the processor 203 through analysis of the pulse rate signal. Furthermore, using a single sensor to measure both pulse rate and motion reduces the complexity of the wearable device.

### Additional Configuration Considerations

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

Certain embodiments are described herein as including logic or a number of components, modules, or mechanisms. Modules may constitute either software modules (e.g., code embodied on a machine-readable medium or in a transmission signal) or hardware modules. A hardware module is tangible unit capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

The various operations of example methods described herein may be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

The one or more processors may also operate to support performance of the relevant operations in a “cloud computing” environment or as a “software as a service” (SaaS). For example, at least some of the operations may be performed by a group of computers (as examples of machines including processors), these operations being accessible via a network (e.g., the Internet) and via one or more appropriate interfaces (e.g., application program interfaces (APIs).)

The performance of certain of the operations may be distributed among the one or more processors, not only residing within a single machine, but deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Some portions of this specification are presented in terms of algorithms or symbolic representations of operations on data stored as bits or binary digital signals within a machine memory (e.g., a computer memory). These algorithms or symbolic representations are examples of techniques used by those of ordinary skill in the data processing arts to convey the substance of their work to others skilled in the art. As used herein, an “algorithm” is a self-consistent sequence of operations or similar processing leading to a desired result. In this context, algorithms and operations involve physical manipulation of physical quantities. Typically, but not necessarily, such quantities may take the form of electrical, magnetic, or optical signals capable of being stored, accessed, transferred, combined, compared, or otherwise manipulated by a machine. It is convenient at times, principally for reasons of common usage, to refer to such signals using words such as “data,” “content,” “bits,” “values,” “elements,” “symbols,” “characters,” “terms,” “numbers,” “numerals,” or the like. These words, however, are merely convenient labels and are to be associated with appropriate physical quantities.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for measuring pulse rate of a user during periods of motion through the disclosed principles herein. Thus, while particular embodiments and applications have been illustrated and described, it is to be understood that the disclosed embodiments are not limited to the precise construction and components disclosed herein. Various modifications, changes and variations, which will be apparent to those skilled in the art, may be made in the arrangement, operation and details of the method and apparatus disclosed herein without departing from the spirit and scope defined in the appended claims.

1. A device configured to be worn by a user during an activity, the device comprising:
an optical pulse rate detector including:
a first emitter to transmit light at a first light output level into body tissue of the user,
a second emitter to transmit light at a second light output level into the body tissue of the user,
a photodetector to receive reflected light from the body tissue and producing a voltage indicative of an amount of the reflected light,
a sensor controller operable to:
drive the first emitter to output the light at the first light output level and the second emitter to output the light at the second light output level, and
sample the voltage produced by the photodetector to generate a first output signal indicative of an amount of the light at the first light output level reflected from the body tissue and a second output signal indicative of an amount of the light at the second light output level reflected from the body tissue, and
an adaptive filter to generate a pulse rate signal by removing the second output signal from the first output signal; and
a processor to determine a pulse rate of the user using the generated pulse rate signal.

2. The device of claim 1, wherein the sensor controller is operable to drive the first emitter to output the light at the first light output level and the second emitter to output the light at the second light output level by further being operable to:
generate a first drive signal to drive the first emitter, the first drive signal comprising a plurality of current pulses; and
generate a second drive signal to drive the second emitter, the second drive signal comprising a plurality of current pulses each generated between consecutive current pulses of the first drive signal.

3. The device of claim 2, wherein the current pulses of the first drive signal have a higher magnitude than the current pulses of the second drive signal.

4. The device of claim 2, wherein the sensor controller adjusts a magnitude of the current pulses of the second drive signal based on a comparison of the second output signal to the first output signal.

5. The device of claim 1, wherein the first emitter transmits light having a wavelength responsive to blood volume.

6. The device of claim 1, wherein the second emitter transmits light having a wavelength responsive to motion of the user.

7. The device of claim 1, wherein the processor further determines at least one of a beat-to-beat variance of the pulse rate, a beat-to-beat magnitude of the pulse rate, a beat-to-beat coherence of the pulse rate, and a respiration rate of the user using the pulse rate signal.

8. An optical pulse rate detector, comprising:
at least one emitter to transmit light at a first light output level into body tissue of a user and transmit light at a second light output level into the body tissue of the user;
a photodetector to receive reflected light from the body tissue and producing a voltage indicative of an amount of the reflected light; and
an adaptive filter to receive from the photodetector, a first output signal indicative of an amount of the light at the first light output level reflected from the body tissue and a second output signal indicative of an amount of the light at the second output level reflected from the body tissue, the adaptive filter configured to generate a pulse rate signal indicative of pulse rate of the user by removing the second output signal from the first output signal.

9. The optical pulse rate detector of claim 8, further comprising a sensor controller to drive the at least one emitter, the sensor controller operable to:
generate a first drive signal to drive the at least one emitter, the first drive signal comprising a plurality of current pulses; and
generate a second drive signal to drive the at least one emitter, the second drive signal comprising a plurality of current pulses each generated between consecutive current pulses of the first drive signal.

10. The optical pulse rate detector of claim 9, wherein the current pulses of the first drive signal have a higher magnitude than the current pulses of the second drive signal.

11. The optical pulse rate detector of claim 9, wherein the sensor controller adjusts a magnitude of the current pulses of the second drive signal based on a comparison of the second output signal to the first output signal.

12. The optical pulse rate detector of claim 8, wherein the at least one emitter transmits light having a wavelength responsive to blood volume.

13. The optical pulse rate detector of claim 12, wherein the at least one emitter comprises a second emitter to transmit light having a wavelength responsive to motion of the user.

14. The optical pulse rate detector of claim 8, wherein the processor further determines at least one of a beat-to-beat variance of the pulse rate, a beat-to-beat magnitude of the pulse rate, a beat-to-beat coherence of the pulse rate, and a respiration rate of the user using the pulse rate signal.

15. A non-transitory computer-readable storage medium storing executable instructions for determining a pulse rate of a user of a wearable device, the wearable device comprising an optical pulse rate detector including at least one emitter, an adaptive filter, and a processor, the instructions when executed by a processor causing the processor to:
transmit by the emitter, light at a first light output level into body tissue of the user; transmit by the emitter, light at a second light output level into the body tissue of the user;
receive at the adaptive filter, a first signal indicative of an amount of the light at the first light output level reflected from the body tissue and a second output signal indicative of an amount of the light at the second light output level;
generate a pulse rate signal indicative of a pulse rate of the user by removing the second output signal from the first output signal; and
determine a pulse rate of the user using the pulse rate signal.

16. The non-transitory computer-readable storage medium of claim 15, further comprising instructions that when executed by the processor cause the processor to:
generate a first drive signal to drive the emitter to output the light at the first light output level, the first drive signal comprising a plurality of current pulses; and
generate a second drive signal to drive the emitter to output the light at the second light output level, the second drive signal comprising a plurality of current pulses each generated between consecutive current pulses of the first drive signal.

17. The non-transitory computer-readable storage medium of claim 16, wherein the current pulses of the first drive signal have a higher magnitude than the current pulses of the second drive signal.

18. The non-transitory computer-readable storage medium of claim 16, further comprising instructions that when executed by the processor cause the processor to adjust a magnitude of the current pulses of the second drive signal based on a comparison of the second output signal to the first output signal.

19. The non-transitory computer-readable storage medium of claim 15, wherein the light at the first light output level has a wavelength responsive to blood volume.

20. The non-transitory computer-readable storage medium of claim 19, wherein the optical pulse rate detector includes a second emitter emitting the light at the second light output level, and wherein the light at the second output level has a wavelength responsive to motion of the user.
